(19) United States
(12) Patent Application Publication
(10) Pub. No.: US 2024/0325452 A1
(43) Pub. Date: Oct. 3, 2024

(54) METHODS AND COMPOSITIONS FOR DREADD-ACTIVATABLE CELLS FOR NEURAL REPAIR

(71) Applicants: Drexel University, Philadelphia, PA (US); The J. David Gladstone Institutes,a testamentary trust established under the Will of J. D. Gladstone, San Francisco, CA (US)

(72) Inventors: Michael Aron Lane, Philadelphia, PA (US); Lyandysha Viktorovna Zholudeva, Concord, CA (US)

(21) Appl. No.: 18/620,126

(22) Filed: Mar. 28, 2024

Related U.S. Application Data
(60) Provisional application No. 63/492,697, filed on Mar. 28, 2023.

(51) Int. Cl. A61K 35/30; A61P 25/00; C07K 14/705; C12N 5/0797

(52) U.S. Cl. CPC A61K 35/30; A61P 25/00; C07K 14/705; C12N 5/0623; C12N 2506/45; C12N 2510/00

(57) ABSTRACT

Compositions and methods for administering DREADD-activatable cells for repair of the nervous system after injury or disease are contemplated herein.

### PRIORITY

This application claims the benefit of U.S. Provisional Application No. 63/492,697, filed Mar. 28, 2023, the content of which is herein incorporated by reference in its entirety.

### STATEMENT REGARDING GOVERNMENT FUNDING

This invention was made with government support under Grant Number NS119348-01A1 awarded by the National Institutes of Health. The government has certain rights in the invention.

### FIELD OF THE INVENTION

This invention relates to the fields of Designer Receptors Exclusively Activated by Designer Drug (DREADD)-activatable cells for repair of the nervous system after injury or disease.

### BACKGROUND OF THE INVENTION

The nervous system is a complex highly specialized network that organizes, processes, interprets, and directs interactions between the person and the world. The nervous system controls a variety of voluntary and involuntary functions. Accordingly, damage to the nervous system causes a variety of problems depending on the area that is damaged. Damage to the nervous system can occur slowly and cause a gradual loss of function or may be caused by a sudden and life-threatening problem or trauma. Symptoms are often severe; however, options for treating this damage are limited.

### SUMMARY OF THE INVENTION

In decades of transplantation work, many different types of cells have been transplanted without having control over them after they are transplanted. These cell therapies rely on spontaneous mechanisms of action from the transplant to cause a therapeutic effect. No other invention has been able to demonstrate a non-invasive, yet direct activation of cells, that have been transplanted into injured tissues. Thus, there is a need for safe and effective pharmacologic treatments capable of transplanting and activating cells for the repair of an injured or diseased nervous system.

Provided herein are engineered, neural progenitor cells comprising an exogenous nucleic acid that encodes a DREADD receptor. In certain embodiments, the DREDD receptor is activated by a DREADD agonist selected from clozapine, clozapine-n-oxide (CNO), J-60, perlapine, Salvinorin B, DREADD agonist 2, J60 dihydrochloride, J52 dihydrochloride or 11-(1-piperazinyl)-5H-dibenzo[b,e][1,4]diazepine. In certain embodiments, the response to the DREADD agonist is reversable.

In another aspect, the present invention comprises methods for repairing a damaged nervous system in a patient in need thereof, the method comprising administering the cells provided herein and activating the response of said cells. In certain embodiments the damage to the nervous system is caused by injury or disease.

In still another aspect, provided herein are methods for treating nervous system injury or disease in a patient in need thereof, the method comprising administering the cells disclosed herein and activating the response of said cells. In certain embodiments the patient has damage to at least one of the brain or the spinal cord. In certain embodiments, the unmodified cells are obtained from at least one of the patient or a donor and used to generate the engineered cells. In another aspect, the unmodified cells are stem cells and are differentiated to neural progenitor cells. In yet another aspect, administration of the cells improves motor recovery of the patient when compared to an untreated control. In certain embodiments, the cells display at least one of anatomical and functional evidence of connectivity.

In one aspect, the cells are administered prior to surgical treatment. In another aspect, the cells are administered after surgical treatment. In certain embodiments, said patient is participating in rehabilitative physical therapy. In certain embodiments, the rehabilitative physical therapy maintains or strengthens muscle function or redevelops fine motor skills. In certain embodiments, the cells are activated weeks or months after administration. In certain embodiments, said response is activated by exposure to at least one DREADD agonist. In certain embodiments, the neural progenitor cell is a V2a Spinal Interneuron.

Still other aspects and advantages of these compositions and methods are described further in the following detailed description.

### BRIEF DESCRIPTION OF THE FIGURES

FIGS. 1A-1B. Timeline for differentiating V2a spinal interneurons from human pluripotent stem cells (hPSCs, FIG. 1A). Subset of co-cultured cells were plated on Multielectrode arrays (MEA) to record spontaneous activity, and their response to clozapine (1 uM) administration. These data show spontaneous neuronal firing (examples shown by arrow heads in recording on the left), that is silenced in the presence of clozapine. (FIG. 1B)

FIGS. 2A-2D. Transplanted DREADD-expressing V2a SpINs survive and differentiate into neurons. V2a SpINs differentiated from human pluripotent stem cells can survive after transplantation into the injured spinal cord (FIG. 2A, red line denotes injury area, transplanted cells shown in pink). High magnification images (FIGS. 2B-2D) of the transplant area denoted by white square in FIG. 2A shows transplanted cells differentiate into neurons. These cells express markers for human cytoplasmic protein (FIG. 2B, STEM121, red) and neuronal marker (FIG. 2C, NeuN, blue) with merge shown in FIG. 2D. Scale bars are 500 microns in FIG. 2A and 100 microns in FIGS. 2B-D.

FIGS. 3A-3B. Inhibitory Effects of Clozapine on DREADD-expressing cells in vivo post-transplantation. (FIG. 3A) Schematic diagram of the transplantation paradigm into the injured cervical spinal cord. Vehicle (Hank's Buffered Salt Solution) or Cells are injected directly into the injury site, 1 week post-injury, and compared at 1-month post-transplantation via diaphragm electromyography. (FIG. 3B) Representative traces of diaphragm electromyography before and after Clozapine and with a hypoxia challenge. Diaphragm activity from vehicle-recipient (top, black traces), progenitor cell recipients with no DREADD expressing cells (middle, green traces) and progenitor cell recipients with DREADD expressing cells (bottom, blue traces). Aqua line indicates increase in activity in vehicle-recipient when undergoing hypoxia challenge, whereas Red lines indicate decrease in activity compared to Pre-Clozapine.

### DETAILED DESCRIPTION OF THE INVENTION

In certain embodiments of this invention, provided herein are DREADD-activatable cells (e.g., neural cells) for repair of the nervous system (e.g., brain and spinal cord) after injury or disease. DREADD-activatable cells refers to cells that express DREADD proteins and are responsive (e.g., increase or decrease their activity) to specific drugs (e.g., clozapine, clozapine-n-oxide, J-60, etc.). A novel strategy for neural repair that involves transplanting donor neural progenitor cells engineered from DREADD stem cells is disclosed herein. In methods described herein, the cells are transplanted as neural progenitor cells, survive the inhibitory environment known to comprise within the injured central nervous system, then weeks to months after transplantation, the cells are controlled (activated or silenced) with specific drugs to elicit function from the transplant.

Emerging technologies, such as cell-based repair strategies, offer new promise for some of the most devastating medical conditions that currently lack treatments. However, to harness the full therapeutic potential of stem cells, it will be necessary to understand how to direct their differentiation to appropriate cell phenotypes and ensure that their phenotype and function persist after transplantation into a pathologic environment.

Composition and methods are provide to repair the injured or diseased central nervous system is novel and gives precise control over when the transplanted cells are active and/or silenced using DREADD-activation. It provides one with control over the transplants themselves.

In certain embodiments, the invention is a biological product intended for transplantation into the injured or diseases nervous system that can be reversibly controlled with specific drugs. This ability enables the entrainment of transplanted cells to perform in a functionally and therapeutically relevant way. These cells are a specific type of neuron that are engineered to be relevant to neural repair.

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

### Definitions

Unless otherwise defined herein, scientific and technical terms used in connection with the present application shall have the meanings that are commonly understood by those of ordinary skill in the art. In addition to definitions included in this section, further definitions of terms are interspersed throughout the text.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, where compositions, components, methods, or steps are described as required in one or more embodiments, additional embodiments are contemplated and are disclosed hereby for fewer compositions, components, methods, or steps, and for fewer compositions, components, methods, or steps in addition to other compositions, components, methods, or steps. All compositions, components, methods, or steps provided herein may be combined with one or more of any of the other compositions, components, methods, or steps provided herein unless otherwise indicated.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

A “sample” refers to a sample from a subject that may be tested. The sample may comprise cells, and it may comprise body fluids, such as blood, serum, plasma, cerebral spinal fluid, urine, saliva, tears, pleural fluid, and the like.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “subject,” “individual,” and “patient” are used interchangeably herein, and refer to an animal, for example a human, to whom treatment, including prophylactic treatment, with the composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term does not denote a particular age or sex. Thus, adult and newborn subjects, as well as fetuses, whether male or female, are intended to be covered.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

The term “pharmaceutically acceptable carrier” or “diluent” is intended to include any and all solvents, dispersion media, coatings, antibacterial and antifungal agents, isotonic and absorption delaying agents, adjuvants and the like, compatible with administration to humans. In one embodiment, the diluent is saline or buffered saline.

The term “modulate” as used herein refers to the ability of a compound to change an activity in some measurable way as compared to an appropriate control. As a result of the presence of compounds in the assays, activities can increase or decrease as compared to controls in the absence of these compounds. For example, an increase in activity is at least 25%, including at least 50%, or at least 100% compared to the level of activity in the absence of the compound. Similarly, a decrease in activity can include at least 25%, including at least 50%, or at least 100% compared to the level of activity in the absence of the compound.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

“Human pluripotent stem cells” or “hPSCs”, which include human embryonic stem cells (hESCs) and human induced pluripotent stem cells (hiPSCs), are cells that can self-renew indefinitely in culture while maintaining the ability to become almost any cell type in the human body. In certain embodiments, the hPSCs are genetically altered to respond a stimulus by specific drugs. These DREADD-activatable cells are then differentiated into DREADD neural progenitor cells.

“Neural progenitor cells (NPCs)” are the progenitor cells of the CNS that give rise to many, if not all, of the glial and neuronal cell types that populate the CNS. NPCs do not generate the non-neural cells that are also present in the CNS, such as immune system cells. NPCs are present in the CNS of developing embryos but are also found in the neonatal and mature adult brain, and therefore are not strictly embryonic stem cells. NPCs are characterized based on their location in the brain, morphology, gene expression profile, temporal distribution and function. In general, embryonic NPCs have more potential than NPCs in the adult brain. In certain embodiments, the primary component of transplantable NPCs is a V2a spinal interneuron.

V2a neurons are a genetically defined cell class that forms a major excitatory descending or ascending pathway from the brainstem reticular formation to the spinal cord and vice versa as well as within the spinal cord. Their activation has been linked to the termination of locomotor and breathing activity based on broad DREADD manipulations. V2a interneurons play a role in both forelimb and hindlimb movements, and two major types have been identified that emerge during development: type I neurons marked by high Chx10 expression throughout and beyond early developmental stages, usually located in lower-levels of spinal cord and type II neurons that downregulate Chx10 expression, typically found in higher-levels, or cervical, spinal cord. Types I and II V2a interneurons are arrayed in counter-gradients, and this network activates different patterns of motor output at cervical and lumbar levels.

V2a interneurons are present at all spinal levels, and anatomical and physiological studies have identified ascending connections to brainstem nuclei and local connections to motor neurons. These interneurons can be identified by their expression of the transcription factor Chx10/Vsx2.

In the ventral spinal cord, V2a interneurons represent locally projecting, candidate pre-motoneurons, involved in the control of left-right coordination of limb movements. In the brainstem, V2a neurons populate all antero-posterior levels of the medullary and pontine reticular formations (RFs) and might have diverse functions and projection profiles. Initially linked to breathing control through local projections in the RF, they also represent a subset of excitatory reticulospinal (RS) neurons that collectively innervate multiple spinal segments. Broad DREADD activation of V2a neurons in the gigantocellular reticular nucleus (Gi) causes an arrest of ongoing locomotion, while broad silencing favors mobility. These effects were attributed to V2a neurons with direct projections to the hindlimb controllers in the lumbar spinal cord. In certain embodiments, the engineered cells are V2a neurons, or V2a neuron-like cells.

The nervous system is the major controlling, regulating and communication system in the body. It controls complicated processes like movement, though and memory and plays an essential role in natural bodily processes. The nervous system affects every aspect of health including, without limitation, thoughts, memory, learning, feelings, movements, balance, coordination, senses, including sight, hearing, taste, touch and feeling, sleep, healing, aging, heartbeat, breathing patterns, stress responses, digestion, and other body processes. The nervous system uses specialized cells called neurons to send signals throughout the body. These signals carry data to and from the brain.

A “damaged nervous system” revers to any disorder or condition that cases an injured nerve. “Injured nerves” refer to any nerve that has trouble sending or receiving a signal. In certain embodiments, the nerves are so damaged that they are completely unable to send or receive signals. Nerve injury can cause numbness, pain, inability to move, and/or death. Other symptoms include, vision problems, headaches, slurred speech, loss of sensation, tremors or tics (random muscle movements), changes in behavior or memory, problems with coordination or movement, and muscle weakness. The nervous system can be damaged by disease, such as cancers, autoimmune diseases like diabetes, lupus and rheumatoid arthritis, multiple sclerosis, stroke, accidental injury, pressure on the nerve, toxic chemicals including certain medicines, and advanced age. In certain circumstances, surgery is required to rectify the damage to the nervous system.

### DREADD Producing Cells

The neural progenitor cells provided herein are modified such that they produce DREADD proteins that are activated by a controlled stimulus at a desirable time. In one embodiment, the cells produce the DREADD protein hMDq, hM4Di, hM2D, kappa opioid receptors (KORs), Gq-coupled DREADDs (G_{q}D), Gi-coupled DREADDs (GᵢD), Gs-coupled DREADDs (GₛD). In one embodiment, these cells are activated by a DREADD agonist, such as clozapine, clozapine-n-oxide (CNO), J-60, perlapine, Salvinorin B, DREADD agonist 2, J60 dihydrochloride, and J52 dihydrochloride and/or 11-(1-piperazinyl)-5H-dibenzo[b,e][1,4]diazepine. Other DREADDs and DREADD ligands are known to those skilled in the art (see e.g., hellobio.com/dreadds-review).

The phrase “Designer Receptors Activated by Designer Drugs” or “DREADDs” refers to a class of chemogenic proteins that are manipulated to react to specific stimulus (receptor-ligand interaction). DREADDs allow researchers to quickly and simply control cellular switches by administering a DREADD agonist. After administration, DREADD agonists have rapid clearance which allows for more precise control over the pathway and easy repetition as required. Additionally, administration of the DREADD agonist can precisely manipulate a large population of cells using multiple injections. In view of these, DREADDs allow for more controlled activation of cells than other techniques. Common DREADDs include, without limitation, hMDq, hM4Di, hM2D, kappa opioid receptors, Gs-coupled DREADDs (GₛD).

DREADD technology uses viral vectors to introduce at least one of these chemogenic proteins into cells of interest. These vectors can be used to introduce the DREADD proteins in vivo or in vitro. As DREADD are usually targeted to specific types of cells, different promoters can be incorporated into the viral gene construct. Once present in the subject, these cells can be activated using at least one DREADD agonist.

The term “agonist” refers to an agent, e.g., ligand, protein, polypeptide, peptide, lipid, antibody, antibody fragment, large molecule, or small molecule that binds to a receptor and has an intrinsic effect such as inducing a receptor-mediated response. For example, the agonist may stimulate, increase, activate, facilitate, enhance, or up regulate the activity of the receptor.

The term “DREADD agonist” refers to an agonist that activates the DREADD proteins in an engineered cell. In certain embodiments, the DREADD agonist is at least one of clozapine, clozapine-n-oxide (CNO), JHU37152 (J-52), JHU37160 (J-60), and 11-(1-piperazinyl)-5H-dibenzo[b,e][1,4]diazepine, and Deschloroclozapine (DCZ), and/or salvinorin (SALB).

### Vectors for Modifying Neural Cells

In certain embodiments, the cells are modified using a vector to contain an expression cassette that encodes the DREADD. As used herein, a “vector” comprises any genetic element including, without limitation, naked DNA, a phage, transposon, cosmid, episome, plasmid, bacteria, or a virus, which expresses, or causes to be expressed, a desired nucleic acid construct. Exemplary constructs which can be used to generate a vector are known to those skilled in the art including plasmid Nos. 178707, 125146, 125147, 121538, 121539, 65418, 65417, 177328, 111397, 44361, 44362, and 135635 available from addgene.org. See e.g. addgene.org/15147, addgene.org/121539, and addgene.org/search/catalog/plasmids/?q=dreadd+viral+vector.

One of skill in the art can readily construct a suitable expression cassette, see, e.g. Smith K S et al. DREADDs: Use and application in behavioral neuroscience. Behav Neurosci. 2016; 130 (2): 137-155 (incorporated herein by reference) and Hu Zhu et al. DREADD: A Chemogenetic GPCR Signaling Platform, International Journal of Neuropsychopharmacology, Volume 18, Issue 1, January 2015, pyu007 (incorporated herein by reference).

In one embodiment, the vector is a non-pathogenic virus. In another embodiment, the vector is a non-replicating virus. In one embodiment, a desirable viral vector may be a retroviral vector, such as a lentiviral vector. In another embodiment, a desirable vector is an adenoviral vector. In still another embodiment, a suitable vector is an adeno-associated viral vector. Adeno, adeno-associated and lentiviruses are generally preferred because they infect actively dividing as well as resting and differentiated cells such as the stem cells, macrophages and neurons. A variety of adenovirus, lentivirus and AAV strains are available from the American Type Culture Collection, Manassas, Va., or available by request from a variety of commercial and institutional sources. Further, the sequences of many such strains are available from a variety of databases including, e.g., PubMed and GenBank.

In one embodiment, a lentiviral vector is used. Among useful vectors are the equine infectious anemia virus and feline as well as bovine immunodeficiency virus, and HIV-based vectors. A variety of useful lentivirus vectors, as well as the methods and manipulations for generating such vectors for use in transducing cells and expressing heterologous genes, e.g., N Manjunath et al, 2009 Adv Drug Deliv Rev., 61 (9): 732-745; Porter et al., N Engl J Med. 2011 Aug. 25; 365 (8): 725-33), among others.

In another embodiment, the vector used herein is an adenovirus vector. Such vectors can be constructed using adenovirus DNA of one or more of any of the known adenovirus serotypes. See, e.g., T. Shenk et al., Adenoviridae: The Viruses and their Replication “, Ch. 67, in FIELD'S VIROLOGY, 6.sup.th Ed., edited by B. N Fields et al, (Lippincott Raven Publishers, Philadelphia, 1996), p. 111-2112; U.S. Pat. No. 6,083,716, which describes the genome of two chimpanzee adenoviruses; U.S. Pat. No. 7,247,472; WO 2005/1071093, etc. One of skill in the art can readily construct a suitable adenovirus vector to carry and express a nucleotide construct as described herein. In another embodiment, the vector used herein is an adeno-associated virus (AAV) vector. Such vectors can be constructed using AAV DNA of one or more of the known AAV scrotypes. See, e.g., U.S. Pat. Nos. 7,803,611; 7,696,179, among others.

In certain embodiments, the construct is delivered via a lipid nanoparticle. The term “lipid nanoparticle” refers to a lipid composition having a typically spherical structure with an average diameter of 10 to 1000 nanometers, e.g. 75 nm to 750 nm, or 100 nm and 350 nm, or between 250 nm to about 500 nm. In some formulations, lipid nanoparticles can comprise at least one cationic lipid, at least one noncationic lipid, and at least one conjugated lipid. Lipid nanoparticles known in the art that are suitable for encapsulating nucleic acids, such as mRNA, may be used. “Average diameter” is the average size of the population of nanoparticles comprising the lipophilic phase and the hydrophilic phase. The mean size of these systems can be measured by standard methods known by the person skilled in the art. Examples of suitable lipid nanoparticles for gene therapy is described, e.g., L. Battaglia and E. Ugazio, J Nanomaterials, Vol 2019, Article ID 283441, pp. 1-22; US2012/0183589A1; and WO 2012/170930 which are incorporated herein by reference in their entirety.

These vectors also include conventional control elements that permit transcription, translation and/or expression of the nucleic acid constructs in a cell transfected with the plasmid vector or infected with the viral vector. A great number of expression control sequences, including promoters which are native, constitutive, inducible and/or tissue-specific, are known in the art and may be utilized. In one embodiment, the promoter is selected based on the chosen vector. In another embodiment, when the vector is lentivirus, the promoter is RSV, U6, H1, CMV IE, EF-1α, ubiquitin C, or phosphoglycero-kinase (PGK) promoter. In one embodiment, the promoter is an RSV promoter. In another embodiment, when the vector is an AAV, the promoter is an RSV, U6, or CMV promoter. In another embodiment, when the vector is an adenovirus, the promoter is RSV, U6, CMV, or HI promoters. In another embodiment, when the vector is Listeria monocytogenes, the promoter is a hly or actA promoter. Still other conventional expression control sequences include selectable markers or reporter genes, which may include sequences encoding geneticin, hygromycin, ampicillin or puromycin resistance, among others. Other components of the vector may include an origin of replication. Selection of these and other promoters and vector elements are conventional and many such sequences are available.

These vectors are generated using the techniques and sequences provided herein, in conjunction with techniques known to those of skill in the art. Such techniques include conventional cloning techniques of cDNA such as those described in texts (Sambrook et al, Molecular Cloning: A Laboratory Manual, Cold Spring Harbor Press, Cold Spring Harbor, N.Y.), use of overlapping oligonucleotide sequences, polymerase chain reaction, and any suitable method which provides the desired nucleotide sequence.

Thus, in one embodiment, using the information taught herein and publicly available and known vector construction components and techniques, one of skill in the art can construct a viral vector (or plasmid) that expresses the desired nucleic acid construct.

In certain embodiments, the vector is a synthetic or artificial viral particle in which an expression cassette containing a nucleic acid sequence encoding the expression control system is packaged in a viral capsid or envelope, where any viral genomic sequences also packaged within the viral capsid or envelope are replication-deficient; i.e., they cannot generate progeny virions but retain the ability to infect target cells. The viral vector can be selected from a lentivirus, adeno-associated virus (AAV), and adenovirus, or a bocavirus.

It should be understood that the compositions in the vector described herein are intended to be applied to other compositions, regiments, aspects, embodiments and methods described across the Specification.

### Components of the Neural Progenitor Differentiation Media

The phrase “mTeSR media” refers to any medium that can be used to maintain hPSC cultures. Adjustments to a mTeSR media will cause differentiation of the hPSC.

The phrase “CHIR” refers to the GSK3β inhibitor CHIR99021, 6-[[2-[[4-(2,4-Dichlorophenyl)-5-(5-methyl-1H-imidazol-2-yl)-2-pyrimidinyl]amino]ethyl]amino]-3-pyridinecarbonitrile. CHIR99021 is an aminopyrimidine derivative. CHIR99021 promotes self-renewal potential of embryonic stem cells (ESCs) of mice by inhibiting glycogen synthase kinase-3 (GSK-3) activity and potentiates the upregulation of β-catenin and c-Myc functions. CHIR99021 promotes self-renewal of ESCs by modulating transforming growth factor β (TGF-β), Notch and mitogen-activated protein kinases (MAPK) signaling pathways. CHIR99021 is an agonist of wingless/integrated (wnt) signaling, upregulates cyclinA expression and promotes cell proliferation in non-small-cell lung cancer (NSCLC) cell lines. In combination with SMAD inhibition (see below), CHIR can be used to derive and maintain primitive neural stem cells of neuromesodermal progenitor identity.

SMADs comprise a family of structurally similar proteins that are the main single transducers for receptors of the TFG-B superfamily, which are critically important for regulating cell development and growth. An “SMAD inhibitor” reduces or decreases the activity or expression of SMAD. Dual SMAD inhibition refers to a process using SMAD inhibitors to rapidly differentiate hPSCs into early neurectoderm. Including but not limited to, dual SMAD inhibition can be achieved with small molecules SB431542 and LDN-193189, both are TGF-beta inhibitors.

“Neural Induction Media” refers to Gibco® PSC Neural Induction Medium or other similar commercially available serum-free medium that provides high efficiency neural induction of human pluripotent stems cells (PSCs).

A Notch Pathway is a highly conserved signaling pathway that is crucial in development and is implicated in malignant transformation. A Notch Pathway inhibitor reduces or decreases the activity of the Notch Pathway. In certain embodiments, the Notch Pathway inhibitor is DAPT.

“BrainPhys Media” refers to the neuronal medium developed by Bardy et al (BrainPhys supports neurophysiological activity, Bardy et al, Proceedings of the National Academy of Sciences May 2015, 112 (20) E2725-E2734; DOI: 10.1073/pnas. 1504393112) and available from Stemcell Technologies (cat #ST-05790). BrainPhys Media simulates various in vivo conditions including: inorganic salt concentration, glucose levels, and osmolarity. Additionally, BrainPhys Media allows for several Neuronal functions including spontaneous and evoked action potentials, spontaneous network calcium dynamics, excitatory synaptic activity, and inhibitory synaptic activity. To sustain cell survival and/or neural differentiation in vitro, supplements such as antioxidants, growth factors, hormones, and proteins can be added to basal media.

“G/BDNF” refers to brain derived neurotropic factor.

“IGF” refers to interferon gamma.

“CNTF” refers to Ciliary Neurotrophic Factor.

### Methods of Generating Activatable Neural Progenitor Cells

Provided herein are methods for generating DREADD-activatable neural progenitor cells for use in the treatment of the nervous system in a subject in need thereof, and compositions containing the same. By DREADD-activatable it is meant that the cells engineered by this method to express at least one DREADD protein and respond to a stimulus, i.e., a DREADD agonist, that either increases or decreases their activity. In one embodiment, the cells produce the DREADD protein hMDq, hM4Di, hM2D, kappa opioid receptors, and/or Gs-coupled DREADDs (GₛD). In certain embodiments this DREADD agonist is selected from a clozapine, clozapine-n-oxide (CNO), J-60, and 11-(1-piperazinyl)-5H-dibenzo[b,e][1,4]diazepine. In certain embodiments, the response to this stimulus is reversable and the cells can be activated or silenced at will. In certain embodiments, the cells are stimulated after several weeks or months after implantation.

In certain embodiments, the methods disclosed herein comprise generating neural progenitor cells from human pluripotent stem cells (hPSCs) obtained from a donor. In certain embodiments the donor is the same person that is treated using the method disclosed herein. In other embodiments, the donor is a healthy subject.

Methods of generating neural progenitor cells from hPSCs are known in the art, and various techniques may be employed. For example, in some embodiments, the cells obtained from the donor are plated in mTeSR media and contacted with (CHIR) and at least two Dual SMAD inhibitor (SMADi). In certain embodiments, the SMADi is selected from LDN-193189 and SB431542. In other embodiments, the SMAD pathway is inhibited using more than one agent, such as SB431542 and dorsomorphin (DM). The cells are then incubated for about 5 days. In certain embodiments, the cells are incubated for about 1-10 days. Further ranges/subranges, and integers within those ranges/subranges, are also contemplated, including but not limited to about 1-5 days, about 1-6 days, about 1-7 days, about 1-8 days, about 1-9 days, about 5-10 days, about 5-9 days, about 5-8 days, about 5-7 days, about 5-6 days, about 3-7 days, about 4-6 days, at least 2 days, at least 3 days, at least 4 days, at least 5 days, at least 6 days, at least 7 days, at least 8 days, at least 9 days, at least 10 days.

The cells are then replated in Neural Induction Medium. Such neural induction media are known in the art, e.g., DMEM/F12, 1% N2 supplement (Thermo Fisher Scientific), 2 μg ml-1 heparin (Sigma-Aldrich) and 1% penicillin/streptomycin. The CHIR and SMADi remain present in the Neural Induction Media and the cells are further contacted with Retinoic Acid (RA). The cells are incubated on this media for about 2 days, then the CHIR and SMADi are removed and the cells are contacted with RA, a Notch Pathway Inhibitor, and purmorphamine (Pur). In certain embodiments the Notch Pathway inhibitor is DAPT. The cells are then allowed to incubate for about 10 days before being re-plated on BrainPhys Media (available from Stem Cell Technologies (cat #ST-05790)) before being administered to the patient. In certain embodiments, while on the BrainPhysMedia, the cells are contacted with G/BDNF, IGF, and CNTF.

In certain embodiments, the cells are replated on BrainPhys media after about 5-15 days. Further ranges/subranges, and integers within those ranges/subranges, are also contemplated, including but not limited to about 5-10 days, about 6-10 days, about 7-10 days, about 8-10 days, about 9-10 days, about 10-15 days, about 11-15 days, about 12-15 days, about 13-15 days, about 14-15 days, about 8-12 days, about 9-11 days, at least 2 days, at least 3 days, at least 4 days, at least 5 days, at least 6 days, at least 7 days, at least 8 days, at least 9 days, at least 10 days.

The person of skill in the art can produce cells according to the methods taught herein, and techniques known in the art, e.g., Butts J C, et al. “V2a interneuron differentiation from mouse and human pluripotent stem cells.” Nat Protoc. 2019 November; 14 (11): 3033-3058. doi: 10.1038/s41596-019-0203-1; and Brown, Chelsea R et al. “Generation of v2a interneurons from mouse embryonic stem cells.” Stem cells and development vol. 23, 15 (2014): 1765-76. doi: 10.1089/scd.2013.0628, which are incorporated herein by reference.

The cells provided herein are modified such that they express at least one DREADD protein. In certain embodiments, the DREADD is at least one of hMDq, hM4Di, hM2D, kappa opioid receptor, or Gs-coupled DREADDs (GₛD). In one aspect of the invention, the cells are modified to express at least one DREADD prior to administration to the patient. In certain embodiments, the cells are modified prior to differentiation into neuronal progenitor cells. In another embodiment, the cells are modified after differentiation into neuronal progenitor cells. In certain embodiments, the cells are modified in vitro.

In one aspect, provided herein, is a vector comprising an engineered nucleic acid sequence encoding at least one DREADD protein that is administered to the target cells before, during, or after differentiation into neural progenitor cells. In certain embodiments, the vector is delivered to the target cell in vitro.

As used herein, the term “target cell” refers to any target cell in which expression of the expression control system. In one embodiment, the target cell is an hPSC.

Vectors useful in are described at length above. In certain embodiments, the vector is a synthetic or artificial viral particle in which an expression cassette containing a nucleic acid sequence encoding the expression control system is packaged in a viral capsid or envelope, where any viral genomic sequences also packaged within the viral capsid or envelope are replication-deficient; i.e., they cannot generate progeny virions but retain the ability to infect target cells. The viral vector can be selected from a lentivirus, adeno-associated virus (AAV), and adenovirus, or a bocavirus.

In certain embodiments, the DREADD protein is activated by a DREADD agonist. In certain embodiments, the DREADD protein is hMDq, hM4Di, hM2D, kappa opioid receptors, and/or Gs-coupled DREADDs (GₛD). In certain embodiments, the DREADD agonist is clozapine, clozapine-n-oxide (CNO), J-60, and 11-(1-piperazinyl)-5H-dibenzo[b,e][1,4]diazepine.

### Methods of Treatment

Provided herein are methods of treatment of damage to the nervous system after injury or disease. The methods include administration of an effective amount of DREADD-activatable neural progenitor cells to a subject in need thereof and activating the response of the cells using a DREADD agonist. In certain embodiments, the patient has damage to the brain and/or the spinal cord. In some embodiments, treatment using the method provided herein improves motor recovery of the patient when compared to an untreated control. In certain embodiments, the cells display anatomical and/or functional evidence of connectivity. In certain embodiments, treatment further comprises at least one additional activation of the cell response.

In certain embodiments, the cells are generated by the method disclosed herein. In certain embodiments, the cells are obtained from a donor. In certain embodiments, the patient is the donor. In other embodiments, the donor is a healthy subject.

In certain embodiments, the method provided herein further comprises patient participation in rehabilitative physical therapy. In certain embodiments, the patient is treated after surgical treatment of the nervous system.

### Cell Administration

The cells described herein may be delivered to a subject by any means. These cells may be administered alone, as pharmaceutical composition in combination with diluents and/or carriers and/or buffers, and other components. In one embodiment, they may be administered in saline. In another embodiment, in a hydrogel. Among other formulations, both saline and hydrogel formulations are contemplated for delivery by injection. Other components may include cytokines, cells, or other agents conventionally used to repair a damaged nervous system. Compositions may include stabilizers, antioxidants, and/or preservatives. Compositions may include salt solutions that have their pH ‘buffered’, e.g., neutral buffered saline or phosphate buffered saline.

The compositions described herein can be formulated for intravenous (IV), intramuscular (IM), intra-articular (IA) and intrathecal (lumbar puncture) administration. The compositions can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

Carriers may include pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. Carriers also includes any and all coatings, antibacterial and antifungal agents, and absorption delaying agents, and the like that are compatible with the activity of the components, e.g., cells, to be delivered, and are physiologically acceptable to the patient. Supplementary active compounds may also be incorporated into the compositions. Pharmaceutically acceptable salt of the compound or molecule useful within the invention.

Other ingredients that may be included are excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Still other additional ingredients that may be included in the pharmaceutical compositions used in the practice of the invention and are known in the art and described elsewhere.

In certain embodiments, the cells described herein are delivered directly to the site of injured tissue. In other embodiments, the cells are delivered via intrathecal administration. In other embodiments, the cells are delivered IV. In other embodiments, the cells are delivered intramuscularly or intra-arterially. The cells may be delivered in one administration, or in several administrations over a course of time.

Effective amounts of the neural progenitor cells and other aspects of a pharmaceutical composition may be determined by one of skill in the art, including by a physician. Such amounts may be determined by with consideration of the age and/or weight of a patient, and further by the size, condition, location, and/or severity of the wound or wounds to be treated. In one embodiment, administration of from 1 to 100 million cells is appropriate. Further ranges/subranges, and integers within those ranges/subranges, are also contemplated, including but not limited to from 1 to 10 million, 1 to 5 million, 1 to 1 million, 1 to 500,000, 1 to 400,000, 1 to 300,000, 1 to 250,000, 1 to 200,000, 1 to 150,000, 1 to 100,000, 1 to 50,000, 1 to 40,000, 1 to 30,000, 1 to 20,000, 1 to 10,000, 1 to 5,000, 1 to 2,500, 1 to 1,000, 1 to 100, 10 million to 100 million, 50 million to 100 million, 75 million to 100 million.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with a damaged nervous system.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

In certain embodiments, the cells described herein are provided with one or more additional therapies for the damaged nervous system. Treatment for includes medications such as those used to promote nerve cell regeneration or to improve the function of existing nerves.

In certain embodiments, the cells described herein are provided with one or more additional therapies for treating a damaged nervous system, including without limitation rehabilitative physical therapy.

Still further provided are kits comprising one of more of the cells described herein in one or more vials, tubes, or other suitable vessels. The kit may further comprise a syringe or other medical instrument suitable to deliver a composition to a subject.

### DREADD-Agonist Activation of Cells

A receptor activated solely by a synthetic ligand (RASSL) or designer receptor exclusively activated by designer drugs (DREADD), is a class of artificially engineered protein receptors used in the field of chemogenetics which are selectively activated by certain ligands. These systems typically utilize G protein-coupled receptors (GPCR) engineered to respond to synthetic ligands, like clozapine N-oxide (CNO), and not to endogenous ligands. Several types of these receptors exists, derived from muscarinic or k-opioid receptors.

One DREADD is based on the human M3 muscarinic receptor (hM3). Only two point mutations of hM3 were required to achieve a mutant receptor with nanomolar potency for CNO, insensitivity to acetylcholine and low constitutive activity and this DREADD receptor was named hM3Dq. MI and M5 muscarinic receptors have been mutated to create DREADDs hM1Dq and hM5Dq respectively.

One commonly used inhibitory DREADD is hM4Di, derived from the M4 muscarinic receptor that couples with the Gi protein. Another Gi coupled human muscarinic receptor, M2, was also mutated to obtain the DREADD receptor hM2D. Another inhibitory Gi-DREADD is the kappa-opioid-receptor (KOR) DREADD (KORD) which is selectively activated by salvinorin B (SalB).

Gs-coupled DREADDs have also been developed. These receptors are also known as GₛD and are chimeric receptors containing intracellular regions of the turkey erythrocyte β-adrenergic receptor substituted into the rat M3 DREADD.

Provided herein are cells that produce at least one DREADD protein and are responsive to a stimulus. In certain embodiments, the DREADD protein is hMDq, hM3Dq (Alexander et al., 2009; Armbruster et al., 2007), hM4Di, hM2D, hM2Di, kappa opioid receptors (KORs), KORD (K-opioid-derived DREADD), and/or Gs-coupled DREADDs (GₛD).

A number of ligands that can be used to activate RASSLs/DREADDs are commercially available. Also, see, for example, Armbruster and Roth, 2005; Armbruster et al., 2007, which are incorporated herein by reference, with particular reference to the DREADD proteins and agonists, such as Gq-DREADD, Gs-DREADD, Gi-DREADD (Armbruster B N, Li X, Pausch M H, Herlitze S, Roth B L. Evolving the lock to fit the key to create a family of G protein-coupled receptors potently activated by an inert ligand. Proc Natl Acad Sci USA. 2007; 104:5163-5168; Armbruster B, Roth B. Creation of Designer Biogenic Amine Receptors via Directed Molecular Evolution. Neuropsychopharmacology. 2005; 30: S265; Alexander G M, Rogan S C, Abbas A I, Armbruster B N, Pei Y, Allen J A, Nonneman R J, Hartmann J, Moy S S, Nicolelis M A, et al. Remote control of neuronal activity in transgenic mice expressing evolved G protein-coupled receptors. Neuron. 2009; 63:27-39; Roth, Neuron. 2016 Feb. 17; 89 (4): 683-694 all of which are incorporated by reference, in particular for DREADDS and DREADDS agonists).

CNO is the prototypical DREADD activator. CNO activates the excitatory Gq-coupled DREADDs: hM3Dq, hM1Dq and hM5Dq and also the inhibitory hM4Di and hM2Di Gi-coupled DREADDs. CNO also activates the Gs-coupled DREADD (GₛD) and the β-arrestin preferring DREADD: rM3Darr (Rq (R165L).

Systemically administered CNO can convert to clozapine which itself activates DREADDs. Clozapine is an atypical antipsychotic which has been indicated to show high DREADD affinity and potency. Subthreshold injections of clozapine itself can be utilized to induce preferential DREADD-mediated behaviors.

DREADD agonist 21, also known as Compound 21, represents an alternative agonist for muscarinic-based DREADDs and an alternative to CNO. It has been reported that Compound 21 has excellent bioavailability, pharmacokinetic properties and brain penetrability and does not undergo reverse metabolism to clozapine. Another known agonist is perlapine, a hypnotic drug for treating insomnia. It acts as an activator of Gq-, Gi-, and Gs DREADDs that has structural similarity to CNO. Another agonist of hM3Dq and hM4Di is deschloroclozapine (DCZ).

SalB B is an activator of KORD.

JHU37160 and JHU37152 have been marketed commercially as novel DREADD ligands, active in vivo, with high potency and affinity for hM3Dq and hM4Di DREADDs.

Diihydrochloride salts of DREADDs ligands that are water-soluble (but with differing stabilities in solution) are also useful in the methods of the invention.

In certain embodiments the stimulus is a DREADD-agonist such as clozapine, clozapine-n-oxide (CNO), JHU37152 (J-52), JHU37160 (J-60), 11-(1-piperazinyl)-5H-dibenzo[b,e][1,4]diazepine, and Deschloroclozapine (DCZ), and/or salvinorin (SALB).

In certain embodiments, the DREADD-agonist is delivered in vivo. In certain embodiments, the DREADD-agonist is delivered using intravenous (IV), intramuscular (IM), intra-articular (IA), intrathecal (e.g., lumbar puncture), or oral administration.

The duration for delivery of the DREADD-agonist is in an amount sufficient to activate the cells without damaging the surrounding tissues. In certain embodiments, the stimulus is provided at the same time that the cells are administered.

In certain embodiments, the stimulus is delivered multiple times after administration of the cells. In certain embodiments, the stimulus is administered at least 1, 2, 3, 4, 5, 6, 7, 8, 9, 10, or more times after the initial stimulus. In certain embodiments, the stimulus is administered one or more times per hour for several hours, days, weeks, or months. In certain embodiments, the stimulus is administered one or more times per day for several days, weeks or months.

In certain embodiments, the cells will be stimulated, by DREADD agonist delivery, after the cells are administered to a patient. In certain embodiments, the stimulus is delivered from 15-21 days after the cells are administered. Further ranges/subranges, and integers within those ranges/subranges, are also contemplated, including but not limited to at least about 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, or 12 months after administration of the cells. In certain embodiments, the DREADD agonist is delivered with the cells.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Differentiating V2a Spinal Interneurons from hPSCs

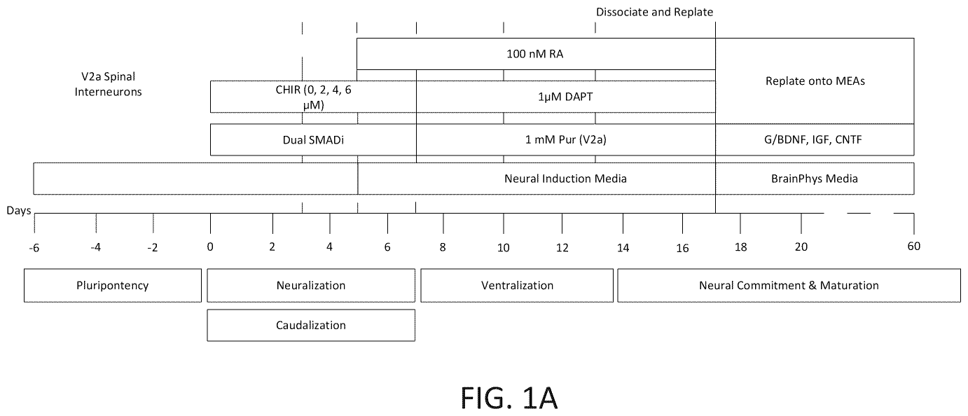

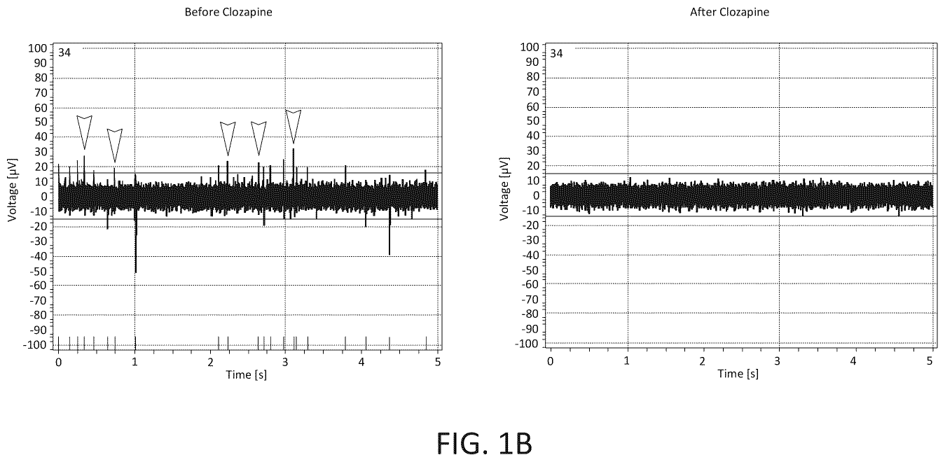

The timeline for differentiating V2a spinal interneurons from hPSCs is seen in FIG. 1A. Donor hPSCs are obtained from a third party and plated on mTeSR media. Cells are then contacted with at least one Dual Smad inhibitor (SMADi) and CHIR for 5 days. In certain embodiments, 0-6 μM, inclusive, of CHIR are added to the media. In certain embodiments, 0, 2, 4, or 6 μM of CHIR are added to the media.

At day 5, the cells are plated on Neural Induction Media and contacted with approximately 100 nM of Retinoic Acid (RA). In certain embodiments, the Neural Induction Media comprises Gibco® PSC Neural Induction Medium or other similar commercially available serum-free medium that provides high efficiency neural induction of human pluripotent stems cells (PSCs). On Day 7, the CHIR and Dual SMADi are removed and replaced with 1 μM of a Notch pathway inhibitor (DAPT) and 1 mM purmorphamine (Pur (V2a)). The cells remain on this media until day 17 where they are replated onto BrainPhys Media and contacted with G/BDNF, IFG, and CNTF. These cells can also be plated onto a microelectrode array (MEA) for further testing. In certain embodiments, the BrainPhys Media is the neuronal medium developed by Bardy et al (BrainPhys supports neurophysiological activity, Bardy et al, Proceedings of the National Academy of Sciences May 2015, 112 (20) E2725-E2734; DOI: 10.1073/pnas.1504393112)

The cells were then tested for neuronal activity. See FIG. 1B. A subset of the differentiated cells were cultured on Multielectrode arrays (MEA) to record spontaneous activity. After 5 seconds, there were several spikes in voltage, indicating that the cells spontaneously fired multiple times. After administration of the DREADD-agonist clozapine (1 uM), no spikes in voltage were observed, indicating that the cells were silenced in the presence of clozapine. These tests indicate successful engineering of DREADD-expressing V2a spinal interneurons (in this case the hM4Di receptor).

### Example 2: Administration and DREADD Expressing V2A Cells

The inhibitory effects of DREADD receptor function can be determined on Chx10-Puro-Gi-DREADD cells in vitro and in vivo. Subjects are anesthetized, and the skull or spinal column is exposed. Engineered cells as generated in Example 1 are delivered using a Hamilton syringe connected to a Narishige micromanipulator. After the injection, the pipette is held in place for 0.5 min before being slowly retracted. For Gi-DREADD receptor activations, 1 uM of a DREADD-agonist (clozapine) was injected at the site of the transplanted cells. Animals can be followed daily after the surgery using behavioral experiments and EMG recordings before, during, and after stimulation by the DREADD-agonist.

### Example 3: Analysis of Inhibitory Effects of Clozapine In Vivo

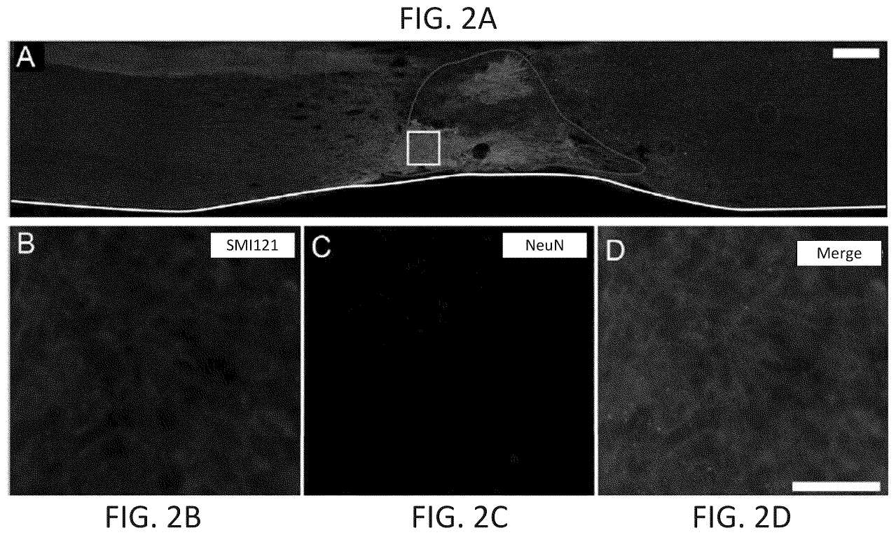

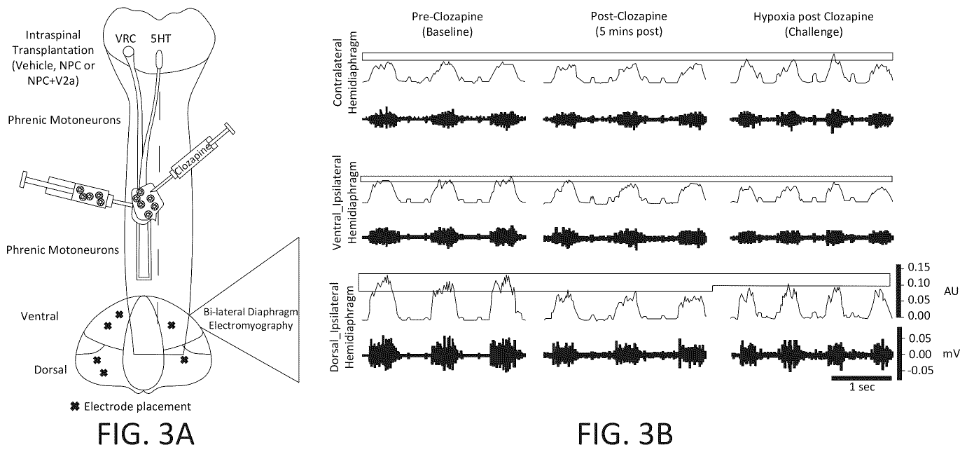

A graphical representation of the transplantation and treatment described herein are shown in FIG. 3A. SpINs have been derived from mouse embryonic stem cells (FIG. 3) and human pluripotent stem cells (FIG. 2). In these examples, the cells were administered to the injured cervical spinal cord of adult rats, at the location of the phrenic motor neurons, and silenced via the DREADD receptor using clozapine. Anatomical assessment of the donor cells, revealed that human derived cells survive and differentiate into mature human neurons (FIGS. 2A-D).

Electrophysiological activity of the diaphragm (FIG. 3B)—innervated by the phrenic network—was recorded from three places on the muscle, before delivery of Clozapine to the transplanted cells, 5 minutes after Clozapine delivery, and after Clozapine with a hypoxia challenge. Quantification of these electrophysiological data reveal that activity from the transplanted neurons not only connect with the injured phrenic network, but delivery of clozapine-attenuating activity in transplanted neurons-resulted in attenuated muscle activity.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

What is claimed is:

1. An engineered, neural progenitor cell comprising an exogenous nucleic acid that encodes a Designer Receptor Activated by Designer Drug (DREADD) receptor.

2. The cell of claim 1, wherein the DREDD receptor is activated by a DREADD agonist selected from clozapine, clozapine-n-oxide (CNO), J-60, perlapine, Salvinorin B, DREADD agonist 2, J60 dihydrochloride, J52 dihydrochloride and 11-(1-piperazinyl)-5H-dibenzo[b,e][1,4]diazepine.

3. The cell of claim 2, wherein DREADD agonist is clozapine.

4. The cell of claim 1, wherein the response to the DREADD agonist is reversable.

5. A method for repairing a damaged nervous system in a patient in need thereof, the method comprising administering the cell of claim 1 and activating the response of said cells.

6. The method of claim 5, wherein the damage to the nervous system is caused by injury or disease.

7. A method for treating nervous system injury or disease in a patient in need thereof, the method comprising administering the cell of claim 1 and activating the response of said cells.

8. The method of claim 5, wherein the patient has damage to at least one of the brain or the spinal cord.

9. The method of claim 5, wherein unmodified cells are obtained from at least one of the patient or a donor and used to generate the engineered cells.

10. The method of claim 9, wherein the unmodified cells are stem cells and are differentiated to neural progenitor cells.

11. The method of claim 7, wherein the treatment improves motor recovery of the patient when compared to an untreated control.

12. The method of claim 7, wherein, after treatment, the administered cells display at least one of anatomical and functional evidence of connectivity.

13. The method of claim 5, wherein administration of the cells occurs prior to surgical treatment.

14. The method of claim 5, wherein administration of the cells occurs after surgical treatment.

15. The method of claim 5, further comprising said patient participating in rehabilitative physical therapy.

16. The method of claim 15, wherein said rehabilitative physical therapy maintains or strengthens muscle function or redevelops fine motor skills.

17. The method of claim 5, wherein said response is activated by exposure to at least one DREADD agonist.

18. The method of claim 5, wherein the cells are activated weeks or months after administration.

19. The method of claim 5, wherein the neural progenitor cell is a V2a Spinal Interneuron.

20. The method of claim 5, wherein the DREADD receptor is selected from hMDq, hM4Di, hM2D or kappa opioid receptor (KORD).
